(19) United States
(12) Patent Application Publication
(10) Pub. No.: US 2006/0084666 A1
(43) Pub. Date: Apr. 20, 2006

(54) Combined treatment with radiation and an epidermal growth factor receptor kinase inhibitor

(76) Inventors: Paul M. Harari, Madison, WI (US); Prakash Chinnaiyan, Madison, WI (US); Shyhmin Jason Huang, Madison, WI (US)

Correspondence Address: OSI PHARMACEUTICALS, INC., 58 SOUTH SERVICE ROAD, MELVILLE, NY, 11747, US

(21) Appl. No.: 11/251,982

(22) Filed: Oct. 17, 2005

Related U.S. Application Data
(60) Provisional application No. 60/619,705, filed on Oct. 18, 2004.

(51) Int. Cl. A61K 31/522; A61N 5/00

(52) U.S. Cl. 514/263.34; 600/1

(57) ABSTRACT

The present invention provides a method for treating tumors or tumor metastases in a patient, comprising administering to the patient a therapeutically effective amount of an EGFR kinase inhibitor, combined with treating the patient simultaneously or sequentially with radiation therapy, with or without additional agents or treatments, such as other anti-cancer drugs. A preferred example of an EGFR kinase inhibitor that can be used in practicing this invention is the compound erlitinib HCl (also known as Tarceva™).

### CROSS-REFERENCE TO RELATED APPLICATIONS

This application claims the benefit of U.S. Provisional Application No. 60/619,705, filed Oct. 18, 2004, which is herein incorporated by reference in its entirety.

### BACKGROUND OF THE INVENTION

The present invention is directed to methods and compositions for treating cancer patients. In particular, the present invention is directed to combined treatment of patients with radiation and an epidermal growth factor receptor (EGFR) kinase inhibitor.

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), and hormone therapy (e.g., tamoxifen, flutamide).

According to the National Cancer Institute, lung cancer is the single largest cause of cancer deaths in the United States and is responsible for nearly 30% of cancer deaths in the country. According to the World Health Organization, there are more than 1.2 million cases worldwide of lung and bronchial cancer each year, causing approximately 1.1 million deaths annually. Almost 90% of lung cancers are caused by smoking. NSCLC is the most common form of lung cancer and accounts for almost 80 percent of all cases. Treatment options for lung cancer are surgery, radiation therapy, and chemotherapy, either alone or in combination, depending on the form and stage of the cancer. For advanced NSCLC, agents that have been shown to be active include cisplatin, carboplatin, paclitaxel, docetaxel, topotecan, irinotecan, vinorelbine, gemcitabine (e.g. gemzar®), and the EGFR kinase inhibitors gefitinib and erlotinib. Cisplatin-containing and carboplatin-containing combination chemotherapy regimens have been shown to produce objective response rates that are higher than those achieved with single-agent chemotherapy (Weick, J. K., et al. (1991) J. Clin. Oncol. 9(7): 1157-1162). It has been reported that paclitaxel has single-agent activity in stage IV patients, with response rates in the range of 21% to 24% (Murphy W. K., et al. (1993) J. Natl. Cancer Inst. 85(5):384-388). Paclitaxel combinations have shown relatively high response rates, significant 1 year survival, and palliation of lung cancer symptoms (Johnson D. H., et al. (1996) J. Clin. Oncol. 14(7):2054-2060). With a paclitaxel plus carboplatin regimen, response rates have been in the range of 27% to 53% with 1-year survival rates of 32% to 54%. However, efficacy of such treatments is such that no specific regimen can be regarded as standard therapy at present.

Head and neck cancers are tumors that arise in the head or neck region, particularly in the nasal cavity, sinuses, lip, mouth, salivary glands, throat, larynx, and in the lymph nodes of the upper neck. Like lung cancer, they are frequently associated with the use of tobacco. Most head and neck cancers are either squamous cell carcinomas or adenocarcinomas. Head and neck cancers account for about 3 percent of all cancers in the United States, and are more common in men and in people over age 50. Treatment for a head and neck cancer depends on a number of factors, including the exact location of the tumor, the stage of the cancer, and the person's age and general health, and can include surgery, radiation therapy and/or chemotherapy.

Over-expression of the epidermal growth factor receptor (EGFR) kinase, or its ligand TGF-alpha, is frequently associated with many cancers, including breast, lung, colorectal and head and neck cancers (Salomon D. S., et al. (1995) Crit. Rev. Oncol. Hematol. 19:183-232; Wells, A. (2000) Signal, 1:4-11), and is believed to contribute to the malignant growth of these tumors. A specific deletion-mutation in the EGFR gene has also been found to increase cellular tumorigenicity (Halatsch, M-E. et al. (2000) J. Neurosurg. 92:297-305; Archer, G. E. et al. (1999) Clin. Cancer Res. 5:2646-2652). Activation of EGFR stimulated signaling pathways promote multiple processes that are potentially cancer-promoting, e.g. proliferation, angiogenesis, cell motility and invasion, decreased apoptosis and induction of drug resistance. The development for use as anti-tumor agents of compounds that directly inhibit the kinase activity of the EGFR, as well as antibodies that reduce EGFR kinase activity by blocking EGFR activation, are areas of intense research effort (de Bono J. S. and Rowinsky, E. K. (2002) Trends in Mol. Medicine 8:S19-S26; Dancey, J. and Sausville, E. A. (2003) Nature Rev. Drug Discovery 2:92-313). Several studies have demonstrated or disclosed that some EGFR kinase inhibitors can improve tumor cell or neoplasia killing when used in combination with certain other anti-cancer or chemotherapeutic agents or treatments (e.g. Shintani, S. et al. (2003) Int. J. Cancer 107:1030-1037; Raben, D. et al. (2002) Semin. Oncol. 29:3746; Herbst, R. S. et al. (2001) Expert Opin. Biol. Ther. 1:719-732; Magne, N et al. (2003) Clin. Can. Res. 9:4735-4732; Magne, N. et al. (2002) British Journal of Cancer 86:819-827; Torrance, C. J. et al. (2000) Nature Med. 6:1024-1028; Gupta, R. A. and DuBois, R. N. (2000) Nature Med. 6:974-975; Tortora, et al. (2003) Clin. Cancer Res. 9:1566-1572; Solomon, B. et al (2003) Int. J. Radiat. Oncol. Biol. Phys. 55:713-723; Krishnan, S. et al. (2003) Frontiers in Bioscience 8, e1-13; Huang, S et al. (1999) Cancer Res. 59:1935-1940; Contessa, J. N. et al. (1999) Clin. Cancer Res. 5:405-411; Li, M. et al. Clin. (2002) Cancer Res. 8:3570-3578; Ciardiello, F. et al. (2003) Clin. Cancer Res. 9:1546-1556; Ciardiello, F. et al. (2000) Clin. Cancer Res. 6:3739-3747; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther. 3:367-380; Bulgaru, A. M. et al. (2003) Expert Rev. Anticancer Ther. 3:269-279; Dancey, J. and Sausville, E. A. (2003) Nature Rev. Drug Discovery 2:92-313; Kim, E. S. et al. (2001) Current Opinion Oncol. 13:506-513; Arteaga, C. L. and Johnson, D. H. (2001) Current Opinion Oncol. 13:491-498; Ciardiello, F. et al. (2000) Clin. Cancer Res. 6:2053-2063; Patent Publication Nos: US 2003/0108545; US 2002/0076408; and US 2003/0157104; and International Patent Publication Nos: WO 99/60023; WO 01/12227; WO 02/055106; WO 03/088971; WO 01/34574; WO 01/76586; WO 02/05791; and WO 02/089842).

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current chemotherapies possess such an ideal profile. Instead, most possess very narrow therapeutic indexes. Furthermore, cancerous cells exposed to slightly sub-lethal concentrations of a chemotherapeutic agent will very often develop resistance to such an agent, and quite often cross-resistance to several other antineoplastic agents as well.

Thus, there is a need for more efficacious treatment for neoplasia and other proliferative disorders. Strategies for enhancing the therapeutic efficacy of existing drugs have involved changes in the schedule for their administration, and also their use in combination with other anticancer or biochemical modulating agents or therapies. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug or therapy given alone).

However, there remains a critical need for improved treatments for lung, head and neck, and other cancers. This invention provides anti-cancer combination therapies that reduce the dosages for individual components or treatments required for efficacy, thereby decreasing side effects associated with each agent or therapy, while maintaining or increasing therapeutic value. The invention described herein provides new therapy combinations, and methods for using therapy combinations in the treatment of lung, head and neck, and other cancers.

### SUMMARY OF THE INVENTION

The present invention provides a method for treating tumors or tumor metastases in a patient, comprising administering to the patient a therapeutically effective amount of an EGFR kinase inhibitor, combined with treating the patient simultaneously or sequentially with radiation therapy, with or without additional agents or treatments, such as other anti-cancer drugs.

A preferred example of an EGFR kinase inhibitor that can be used in practicing this invention is the compound erlitinib HCl (also known as Tarceva™).

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1: Impact of erlotinib on cell cycle phase distribution. UM-SCC6 (A/C) and H226 (B/D) cells were cultured±erlotinib (0.1 μM) for 48 hrs, followed by exposure to XRT (6 Gy). Cells were subsequently stained with propidium iodide and cell cycle distribution was determined by flow cytometry evaluation of DNA content. The overall impact of erlotinib on S-phase fraction following XRT is described in (C, D). Data represents mean values of duplicate samples.

FIG. 2: Effect of erlotinib on radiation-induced apoptosis. (A) Apoptosis was analyzed by fluorescence spectroscopy using pan-caspase inhibitor FAM-VAD-FMK as described in “Material and Methods.” Cells were exposed to erlotinib (1.0 μM×48 hrs), XRT (6 Gy), or the combination. Data represents mean values of two independent experiments. (B) Western blot analysis on whole cell lysates determining cleavage of the poly(ADP-ribose) polymerase (PARP). Cells ±30 minute pre-treatment with erlotinib (1 μM) were harvested 10 and 24 hours after irradiation (6 Gy).

FIG. 3: Effect of erlotinib on EGFR activation following radiation exposure. Indicated cell lines ±24 hr pre-treatment with erlotinib (1.0 μM) were harvested 48 hrs. after exposure to radiation (2 and 10 Gy). Whole cell lysates were evaluated for activated and total EGFR levels.

FIG. 4: Effect of erlotinib on radiation-induced Rad51 expression. UM-SCC6 (A) and H226 (B) cells were either exposed to erlotinib (1.0 μM), XRT (6 Gy), or both in combination and harvested at indicated times. Whole cell lysates were evaluated for Rad51 expression.

FIG. 5: Effect of erlotinib on radiosensitivity. The influence of erlotinib on radiosensitivity was examined by clonogenic survival in UM-SCC1 (A) and H226 (B) cells after exposure to various doses of radiation as described in “Materials and Methods.” Cells were exposed to erlotinib (0.1 μM) for 3 days before irradiation. Control curves were exposed to radiation without erlotinib treatment. Data represents mean values from two independent experiments.

FIG. 6: Antitumor activity of erlotinib in combination with radiation in NSCLC and HNSCC xenografts. H226 (10⁶) cells or UM-SCC6 (10⁶) cells were injected s.c. were injected s.c. into the flank of athymic mice as described in “Materials and Methods.” Mice were either treated with erlotinib (0.8 mg via daily oral gavage), XRT (single 2 Gy fraction twice per week), or both in combination for 3 weeks. Values represent mean tumor size (mm³; n=6/group).

FIG. 7: Effect of erlotinib on the expression of PCNA and p-EGFR after radiation. Immunohistochemical staining was determined using representative human H226 tumor tissue sections taken from mice treated with radiation (XRT) alone, erlotinib alone, or both in combination. Positive (red/brown) staining indicates expression of PCNA and p-EGFR.

FIG. 8: cDNA Microarray analysis of genes differentially regulated by erlotinib (1.0 μM×24 hrs) followed by radiation (6 Gy) in UM-SCC6. Color intensity is assigned to ratios of gene expression; shades of red represents genes that are up-regulated; shades of green, genes that are down-regulated; black, genes that are unchanged. Genes in boldface represents those validated by quantitative RT-PCR and/or western blot analysis.

FIG. 9: Microarray validation of selected genes differentially regulated by erlotinib (1.0 μM×24 hrs) followed by radiation (6 Gy) in UM-SCC6 using quantitative SYBR green RT-PCR (A) and western blot analysis (B). RT-PCR was performed on each sample in duplicate and the ratio was calculated relative to the housekeeping genes hydroxymethylbilane synthase (HMBS) and glyceraldehyde-3 phosphate dehydrogenase (GAPD).

### DETAILED DESCRIPTION OF THE INVENTION

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The data presented in the Examples herein below demonstrate that co-administration an EGFR kinase inhibitor with radiation treatment is effective for treatment of advanced cancers, such as lung cancer, or head and neck cancer. Accordingly, the present invention provides a method for treating tumors or tumor metastases in a patient, comprising administering to the patient a therapeutically effective amount of an EGFR kinase inhibitor, combined with treating the patient simultaneously or sequentially with radiation therapy. In a preferred embodiment of the method, the patient is treated sequentially with radiation therapy after prior treatment by administering to the patient a therapeutically effective amount of an EGFR kinase inhibitor. In an alternative preferred embodiment of the method, the patient is treated sequentially with radiation therapy after prior treatment by administering to the patient a sub-therapeutically effective amount of an EGFR kinase inhibitor. In another alternative preferred embodiment of the method, the patient is treated sequentially with a sub-therapeutically effective amount of radiation therapy after prior treatment by administering to the patient a therapeutically effective amount of an EGFR kinase inhibitor. In one embodiment the tumors or tumor metastases to be treated are lung cancer or head and neck cancer tumors or tumor metastases.

The source of radiation of this invention can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention. In other words, the inhibition of tumor growth by means of the radiation treatment of the combination of this invention is enhanced when combined with treatment using an EGFR kinase inhibitor. Parameters of adjuvant radiation therapies are, for example, contained in International Patent Publication WO 99/60023. Further details of the methodology of radiation treatment of cancer patients is well known to those of skill in the art, and is readily available from the extensive literature in this area (e.g. Principles and Practice of Radiation Oncology (2003), 4^{th} Edition, ISBN 0-7817-35254, ed. Perez C. A. et al., Lippincott Williams and Wilkins; Radiotherapy for head and Neck Cancers (2002), 2^{nd} Edition, ISBN 0-7817-2650-6, Ang, K. K. and Garden, A. S., Lippincott Williams and Wilkins; Principles and Practice of Oncology (2001), 6^{th} Edition, ISBN 0-7817-2387-6, ed. DeVita, V. T. et al., Lippincott Williams and Wilkins).

The present invention further provides a method for treating tumors or tumor metastases in a patient, comprising administering to the patient a therapeutically effective amount of an EGFR kinase inhibitor, combined with treating the patient simultaneously or sequentially with radiation therapy, wherein the radiation treatment is with a radiopharmaceutical, or includes use of a radiopharmaceutical.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition, one or more other cytotoxic, chemotherapeutic or anti-cancer agents, or compounds that enhance the effects of such agents.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. cytoxan®), chlorambucil (CHL; e.g. leukeran®), cisplatin (CisP; e.g. platinol®) busulfan (e.g. myleran®), melphalan, carmustine (BCNU), streptozotocin, triethylenemelamine (TEM), mitomycin C, and the like; anti-metabolites, such as methotrexate (MTX), etoposide (VP16; e.g. vepesid®), 6-mercaptopurine (6MP), 6-thiocguanine (6TG), cytarabine (Ara-C), 5-fluorouracil (5-FU), capecitabine (e.g.Xeloda®), dacarbazine (DTIC), and the like; antibiotics, such as actinomycin D, doxorubicin (DXR; e.g. adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. taxol®) and pactitaxel derivatives, the cytostatic agents, glucocorticoids such as dexamethasone (DEX; e.g. decadron®) and corticosteroids such as prednisone, nucleoside enzyme inhibitors such as hydroxyurea, amino acid depleting enzymes such as asparaginase, leucovorin, folinic acid, raltitrexed, and other folic acid derivatives, and similar, diverse antitumor agents. The following agents may also be used as additional agents: arnifostine (e.g. ethyol®), dactinomycin, mechlorethamine (nitrogen mustard), streptozocin, cyclophosphamide, lornustine (CCNU), doxorubicin lipo (e.g. doxil®), gemcitabine (e.g. gemzar®), daunorubicin lipo (e.g. daunoxome®), procarbazine, mitomycin, docetaxel (e.g. taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon alpha, interferon beta, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, and chlorambucil.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. Fareston®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as Zoladex® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g Antide®, Ares-Serono); the LHRH antagonist ganirelix acetate; the steroidal anti-androgens cyproterone acetate (CPA) and megestrol acetate, commercially available as Megace® (Bristol-Myers Oncology); the nonsteroidal anti-androgen flutamide (2-methyl-N-[4, 20-nitro-3-(trifluoromethyl)phenylpropanamide), commercially available as Eulexin® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. Avastin™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. Vitaxin®); factors such as IFN-alpha (U.S. Pat. Nos. 41530,901, 4,503,035, and 5,231,176); angiostatin and plasminogen fragments (e.g. kringle 14, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloprotienase 2) inhibitors and MMP-9 (matrix-metalloprotienase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition one or more tumor cell pro-apoptotic or apoptosis-stimulating agents.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition one or more signal transduction inhibitors.

Signal transduction inhibitors include, for example: erbB2 receptor inhibitors, such as organic molecules, or antibodies that bind to the erbB2 receptor, for example, trastuzumab (e.g. Herceptin®); inhibitors of other protein tyrosine-kinases, e.g. imitinib (e.g. Gleevec®); ras inhibitors; raf inhibitors; MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

The present invention further thus provides a method for treating tumors or tumor metastases in a patient, comprising administering to the patient a therapeutically effective amount of an EGFR kinase inhibitor, combined with treating the patient simultaneously or sequentially with radiation therapy, and in addition an anti-HER2 antibody or an immunotherapeutically active fragment thereof.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition one or more additional anti-proliferative agents.

Additional antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition a COX II (cyclooxygenase II ) inhibitor. Examples of useful COX-II inhibitors include alecoxib (e.g. Celebrex™), valdecoxib, and rofecoxib.

The present invention further provides a method for treating tumors or tumor metastases in a patient, comprising administering to the patient a therapeutically effective amount of an EGFR kinase inhibitor, combined with treating the patient simultaneously or sequentially with radiation therapy, and in addition treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

The present invention further provides a method for reducing the side effects caused by the treatment of tumors or tumor metastases in a patient with an EGFR kinase inhibitor or radiation, comprising administering to the patient a therapeutically effective amount of an EGFR kinase inhibitor, combined with treating the patient simultaneously or sequentially with radiation therapy, using amounts of inhibitor and radiation that are effective to produce an additive, or a superadditive or synergistic antitumor effect, and that are effective at inhibiting the growth of the tumor.

The present invention further provides a method for the treatment of cancer, comprising administering to a subject in need of such treatment (i) an effective first amount of an EGFR kinase inhibitor, or a pharmaceutically acceptable salt thereof; and (ii) an effective second amount of radiation treatment.

The present invention also provides a method for the treatment of cancer, comprising administering to a subject in need of such treatment (i) a sub-therapeutic first amount of an EGFR kinase inhibitor, or a pharmaceutically acceptable salt thereof; and (ii) an effective second amount of radiation treatment.

The present invention also provides a method for the treatment of cancer, comprising administering to a subject in need of such treatment (i) an effective first amount of an EGFR kinase inhibitor, or a pharmaceutically acceptable salt thereof; and (ii) a sub-therapeutic second amount of radiation treatment.

The present invention also provides a method for the treatment of cancer, comprising administering to a subject in need of such treatment (i) a sub-therapeutic first amount of the EGFR kinase inhibitor erlotinib, or a pharmaceutically acceptable salt thereof; and (ii) a sub-therapeutic second amount of radiation treatment.

In the preceding methods the order of administration of the first and second amounts can be simultaneous or sequential, i.e. radiation treatment can be administered before the EGFR kinase inhibitor, after the EGFR inhibitor, or at the same time as the EGFR kinase inhibitor. In a preferred embodiment the EGFR kinase inhibitor is administered before radiation treatment.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

As used herein, the term “patient” preferably refers to a human in need of treatment with an EGFR kinase inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an EGFR kinase inhibitor.

In a preferred embodiment, the patient is a human in need of treatment for cancer, or a precancerous condition or lesion. The cancer is preferably any cancer treatable, either partially or completely, by administration of an EGFR kinase inhibitor. The cancer may be, for example, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelia] dysplasia, adenomatous dysplasia, hereditary nonpolyposis colon cancer syndrome (HNPCC), Barrett's esophagus, bladder dysplasia, and precancerous cervical conditions.

For purposes of the present invention, “co-administration of” and “co-administering“an EGFR kinase inhibitor with a second agent or compound (e.g. a cytotoxic, chemotherapeutic, or anti-cancer agent) (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The second agent can be administered prior to, at the same time as, or subsequent to administration of the EGFR kinase inhibitor, or in some combination thereof. Where the EGFR kinase inhibitor is administered to the patient at repeated intervals, e.g., during a standard course of treatment, the second agent can be administered prior to, at the same time as, or subsequent to, each administration of the EGFR kinase inhibitor, or some combination thereof, or at different intervals in relation to the EGFR kinase inhibitor treatment, or in a single dose prior to, at any time during, or subsequent to the course of treatment with the EGFR kinase inhibitor.

The EGFR kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the EGFR kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of EGFR kinase inhibitor being used (e.g., small molecule, antibody, RNAi or antisense construct), and the medical judgement of the prescribing physician as based, e.g., on the results of published clinical studies.

The amount of EGFR kinase inhibitor administered and the timing of EGFR kinase inhibitor administration will depend on the type (species, gender, age, weight, etc.) and condition of the patient being treated, the severity of the disease or condition being treated, and on the route of administration. For example, small molecule EGFR kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based EGFR kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The EGFR kinase inhibitors and a second agent can be administered either separately or together by the same or different routes, and in a wide variety of different dosage forms. For example, the EGFR kinase inhibitor is preferably administered orally or parenterally. Where the EGFR kinase inhibitor is erlotinib HCl (Tarceva™), oral administration is preferable.

The EGFR kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The EGFR kinase inhibitor and a second agent can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous EGFR kinase inhibitors should be selected so as to avoid denaturation and/or degradation and loss of biological activity of the inhibitor.

Methods of preparing pharmaceutical compositions comprising an EGFR kinase inhibitor are known in the art, and are described, e.g. in International Patent Publication No. WO 01/34574. In view of the teaching of the present invention, methods of preparing pharmaceutical compositions comprising both an EGFR kinase inhibitor and a second agent will be apparent from the above-cited publications and from other known references, such as Remington's Pharmaceutical Sciences, Mack Publishing Company, Easton, Pa., 18^{th} edition (1990).

For oral administration of EGFR kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous EGFR kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising either an EGFR kinase inhibitor or a second agent in about 0.1% (w/v) to about 5% (w/v) concentration can be prepared.

For veterinary purposes, the active agents can be administered separately or together to animals using any of the forms and by any of the routes described above. In a preferred embodiment, the EGFR kinase inhibitor is administered in the form of a capsule, bolus, tablet, liquid drench, by injection or as an implant. As an alternative, the EGFR kinase inhibitor can be administered with the animal feedstuff, and for this purpose a concentrated feed additive or premix may be prepared for a normal animal feed. The second agent is preferably administered in the form of liquid drench, by injection or as an implant. Such formulations are prepared in a conventional manner in accordance with standard veterinary practice.

The present invention further provides a kit comprising a single container comprising both an EGFR kinase inhibitor and a second agent. The present invention further provides a kit comprising a first container comprising an EGFR kinase inhibitor and a second container comprising a second agent. In a preferred embodiment, the kit containers may further include a pharmaceutically acceptable carrier. The kit may further include a sterile diluent, which is preferably stored in a separate additional container. The kit may further include a package insert comprising printed instructions directing the use of the combined treatment as a method for treating cancer.

As used herein, the term “EGFR kinase inhibitor” refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGFR receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors that include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl) amine (also known as OSI-774, erlotinib, or Tarceva™ (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or Iressa™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, Tarceva™), or other salt forms (e.g. erlotinib mesylate).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or Erbitux™; Imclone Systems), ABX-EGF (Abgenix), EMD 72000 (Merck KgaA, Darmstadt), RH3 (York Medical Bioscience Inc.), and MDX447 (Medarex/Merck KgaA).

Additional antibody-based EGFR kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against EGFR can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-EGFR single chain antibodies. Antibody-based EGFR kinase inhibitors useful in practicing the present invention also include anti-EGFR antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to EGFR.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-EGFR antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

The invention also encompasses a pharmaceutical composition that is comprised of an EGFR kinase inhibitor and a second agent in combination with a pharmaceutically acceptable carrier. This pharmaceutical composition can be used in the methods of the invention described herein for treatment of a patient with an EGFR kinase inhibitor combined with radiation therapy.

Preferably the composition is comprised of a pharmaceutically acceptable carrier and a non-toxic therapeutically effective amount of an EGFR kinase inhibitor compound and a second agent (including pharmaceutically acceptable salts of each component thereof).

Moreover, within this preferred embodiment, the invention encompasses a pharmaceutical composition for the treatment of disease, the use of which results in the inhibition of growth of neoplastic cells, benign or malignant tumors, or metastases, comprising a pharmaceutically acceptable carrier and a non-toxic therapeutically effective amount of an EGFR kinase inhibitor compound and a second agent (including pharmaceutically acceptable salts of each component thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The pharmaceutical compositions of the present invention comprise an EGFR kinase inhibitor compound and a second agent (including pharmaceutically acceptable salts of each component thereof) as active ingredient, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by an EGFR kinase inhibitor compound and a second agent in combination (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an EGFR kinase inhibitor compound and a second agent in combination (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and an EGFR kinase inhibitor compound and a second agent in combination (including pharmaceutically acceptable salts of each component thereof). An EGFR kinase inhibitor compound and a second agent in combination (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise an EGFR kinase inhibitor compound in combination with an anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing the combination of an EGFR kinase inhibitor compound with a second agent (including pharmaceutically acceptable salts of each component thereof) of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an EGFR kinase inhibitor compound and a second agent in combination (including pharmaceutically acceptable salts of each component thereof) may also be prepared in powder or liquid concentrate form.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Introduction

The epidermal growth factor receptors (EGFR) belongs to the ErbB family consisting of four closely related cell membrane receptors: EGFR (HER1 or ErbB1), ErbB2 (HER2), ErbB3 (HER3), and ErbB4 (HER4) (Yarden, Y., et al. (2001) Nat. Rev. Mol. Cell Biol. 2(2): 127-137). Increased expression of EGFR has been observed in a wide variety of tumors, including non-small cell lung cancer (NSCLC) and SCC (squamous cell carcinoma) of the H&N (head and neck) (Grandis, J. R. and Tweardy, D. J. (1993) Cancer Res. 53(15):3579-3584; Rusch, V. et al. (1993) Cancer Res. 53(10 Suppl):2379-2385). Activation of the EGFR signal transduction pathway has been shown to enhance cellular processes involved in tumor growth and progression, including the promotion of proliferation, angiogenesis, invasion, and metastasis (Yarden, Y., et al. (2001) Nat. Rev. Mol. Cell Biol. 2(2): 127-137). Increased expression of EGFR has been correlated with disease progression and poor overall clinical outcome (Maurizi, M., et al. (1996) Br. J. Cancer 74(8):1253-1257; Grandis, J. R., et al. (1998) J. Natl. Cancer Inst. 90(11):824-832). Further, a positive correlation has been described between EGFR expression and tumor resistance to chemotherapy and radiation therapy (Wosikowski, K., et al. (1997) Clin. Cancer Res. 3(12 Pt 1):2405-2414; Ang, K. K., et al. (2002) Cancer Res 62(24):7350-7356).

A broad spectrum of in vitro and in vivo studies have demonstrated the potential for targeting the EGFR in cancer treatment (Moyer, J. D., et al. (1997) Cancer Res 57(21):4838-4848). Erlotinib (Tarceva™,OSI-774) is an EGFR-selective tyrosine kinase inhibitor (TKI) that blocks signal transduction pathways implicated in cancer cell proliferation, survival, and other host-dependent processes promoting cancer progression. It inhibits the activity of purified EGFR TK and EGFR autophosphorylation in intact tumor cells, with 50% inhibitory concentration values of 2 and 20 nmol/L, respectively (Moyer, J. D., et al. (1997) Cancer Res 57(21):4838-4848). Erlotinib is under investigation in clinical trials targeting a variety of tumor sites, both used as single agent, as well as in combination with chemotherapy and/or radiation (Sandler, A. (2003) Oncology 17(11 Suppl. 12): 17-22; Hidalgo, M. (2003) Oncology 17(11 Suppl. 12):11-6; Hidalgo, M., et al. (2003) Semin. Oncol. 30(3 Suppl. 7):25-33; Hidalgo, M., et al. (2001) J. Clin. Oncol. 19(13):3267-79; Soulieres,D., et al. (2004) J. Clin. Oncol. 22(1):77-85; Malik, S. N., et al. (2003) Clin. Cancer Res 9(7):2478-86; Malik, S. N., et al. (2003) Clin. Cancer Res 9(7):2478-86; Grunwald, V., and Hidalgo M. (2003) J. Natl. Cancer Inst. 95(12):851-67).

A series of recently published studies demonstrate strong preclinical evidence regarding the capacity of EGFR inhibition to enhance the anti-tumor activity of ionizing radiation, although the mechanism underlying these processes have not been fully characterized (Huang, S. M., et al. (1999) Cancer Res. 59(8):1935-40; Huang, S. M., et al. (2002) Cancer Res. 62(15):4300-6; Milas, L. et al. (2000) Clin Cancer Res. 6(2):701-8; Bianco, C., et al. (2002) Clin Cancer Res. (10):3250-8; Raben, D., et al. (2002) Semin. Oncol. 29(1 Suppl 4):37-46). This study examines the in vitro and in vivo capacity of the EGFR TKI erlotinib to modulate radiation response in human tumor cell lines and xenografts. The results suggest strong potential for mechanistic synergy between EGFR inhibition and radiation response at several levels, including cell cycle kinetics, apoptosis induction, and the targeting of accelerated cellular repopulation. The potential relationship between EGFR signaling and DNA damage repair is strengthened by new data regarding the inhibition of Rad51 expression by erlotinib. To gain further insight regarding the influence of EGFR signaling on radiation response, microarray studies were performed to examine differential gene regulation. Several promising leads linking EGFR signaling and radiation response involving genes regulating cell structure, adhesion, apoptosis and tumor angiogenesis are identified.

Materials and Methods

Reagents

Cell culture media were obtained from Life Technologies Inc. (Gaithersburg, Md.). Primary antibodies against EGFR and EGR-1 C-19 were obtained from Santa Cruz Biotechnology, Inc. (Santa Cruz, Calif.); pEGFR-1068 was obtained from Cell Signaling Technologies (Beverly, Mass.); PCNA and Rad51 were obtained from Neomarker (Freemont, Calif.); and α-tubulin was obtained from Oncogene Research Products (Cambridge, Mass.). ECL+ chemiluminescence detection system was purchased from Amersham (Arlington Heights, Ill.). All other chemicals were purchased from Sigma Chemical Co. (St. Louis, Mo.). Erlotinib was generously provided by OSI Pharmaceuticals (Melville, N.Y.). All other chemicals were purchased from Sigma Chemical Co. (St. Louis, Mo.).

Cell lines

Human H226 and DU145 cells were obtained from the American Type Culture Collection (Rockville, Md.) and maintained in complete culture media consisting of RPMI (7.4) supplemented with 10% fetal bovine serum and 1% each of penicillin and streptomycin. The UM-SCC1 (floor of mouth) and UM-SCC6 (base of tongue) cell lines were provided by Dr. Thomas E. Carey (University of Michigan) and maintained in complete culture media consisting of DMEM (7.4) supplemented with 10% fetal bovine serum, 1% hydrocortisone, and 1% each of penicillin and streptomycin.

Cell Cycle Analysis.

Cells were harvested after 72 hours exposure of erlotinib, 24 hours exposure to 6 Gy radiation, or in combination. Cell were harvested by trypsinization, washed with PBS, then fixed in 95% ethanol, and stored at 4° C. for up to 7 days before DNA analysis. After removal of ethanol by centrifugation, cells were then incubated with phosphate-citric acid buffer [0.2 M Na₂HPO₄ (pH 7.8), and 4 mM citric acid] at room temperature for 45 min. After centrifugation, cells were then stained with a solution containing 33 μg/ml PI (propidium iodide), 0.13 mg/ml RNase A, 10 mM EDTA, and 0.5% Triton X-100 at 4° C. for 24 hours. Stained nuclei were analyzed for DNA-PI fluorescence using a Becton Dickinson FACScan flow cytometer. Resulting DNA distributions were analyzed by Modfit (Verity Software House, Inc., Topsham, Me.) for the proportion of cells in sub-G0, G1, S, and G2-M phases of the cell cycle.

Apoptosis by Fluorescein Labeled Caspase Inhibitors.

Cells were seeded in 100-mm dishes at a density of 6×10⁵ cells per plate and upon treatment, were harvested by trypsinization, centrifuged, and the cell pellet re-suspended to a final concentration of 2×10⁶ cells/ml. Caspase activity was analyzed by fluorescence spectroscopy according to the manufacturer's protocol (Chemicon International). Briefly, the 300 μL of cells were incubated with 1× Fluorescein labeled pan-caspase inhibitor FAM-VAD-FMK (Ekert, P. G., et al. (1999) Cell Death Differ. 6(11):1081-6) at 37° C. for 1 hr in a humidified atmosphere of 5% CO₂. Cells were then washed twice with wash buffer, finally resuspended in 320 μL PBS. A 100 μL aliquot of the cell suspension was transferred to a black 96-well plate in triplicate. Fluorescence was analyzed on a SpectraMax fluorescence plate reader at 550 nM excitation and 600 nM emission wavelengths.

Immunoblot Analysis.

Following treatment, cells were lysed with RIPA buffer and sonicated in complete proteinase inhibitor cocktail (Roche) and sodium orthovanadate. Fifteen μg of protein extracts were mixed with SDS sample buffer and electrophoresed onto a 10% SDS-polyacrylamide gel under reducing conditions. The separated proteins were transferred onto nitrocellulose membranes (Amersham Pharmacia Biotech, Piscataway, N.J.). The membrane was incubated for 1 hour in blocking buffer (Tris-buffered saline with 0.1% Tween (TBS-T) and 5% nonfat dry milk). The membranes were then incubated with specific primary antibodies. After washing three times with TBS-T buffer, the membrane was incubated with horseradish peroxidase-linked secondary antibody (Amersham Pharmacia Biotech, Piscataway, N.J.) at 1:5000 dilution for 1 hour at room temperature. The signals were visualized with the ECL+ detection system and autoradiography.

For α-tubulin western blots, the antibody probed membranes were stripped with Western Re-Probe buffer (Geno-tech, St. Louis, Mo.) and blocked in Tris-buffered saline with 0.1% Tween (TBS-T) with 5% nonfat dry milk and incubated with rabbit anti-α-tubulin antibody.

Radiation Survival.

Survival following radiation exposure was defined as the ability of the cells to maintain their clonogenic capacity and to form colonies. Briefly, after exposure to radiation, cells were trypsinized, counted and seeded for colony formation in 35-mm dishes at 50-5000 cells/dish. After incubation intervals of 14-21 days, colonies were stained with crystal violet and manually counted. Colonies consisting of 50 cells or more were scored, and 5 replicate dishes containing 10-150 colonies/dish were counted for each treatment. Experiments were performed in duplicate.

Assay for Tumor Growth in Athymic Nude Mice.

In vivo studies were performed as described previously (Huang, S. M., and Harari P M. (2000) Clin. Cancer Res. (6):2166-74). Briefly, UM-SCC1 and H226 cells (˜1×106) were injected s.c. into the flank area of athymic nude mice on day 0. Animal experiments included four treatment groups: control, radiation alone (2 Gy per fraction), erlotinib alone (0.8 mg/day), and radiation in combination with erlotinib. Erlotinib was administered by oral gavage once daily for 3 weeks. Radiation treatment was delivered twice a week for 3 weeks using custom mouse jigs designed to expose only the tumor bed to radiation.

Immunohistochemical Determination of PCNA and pEGFR.

The expression of proliferative and phosphorylated EGFR were detected in histological sections of H226 xenografts as described previously (Huang, S. M., and Harari P M. (2000) Clin. Cancer Res. (6):2166-74). Briefly, excised tumor specimens were fixed in 10% neutral-buffered formalin. After embedding in paraffin, 5-mm sections were cut, and tissue sections were mounted. Sections were dried, deparaffinized, and rehydrated. After quenching endogenous peroxidase activity and blocking nonspecific binding sites, slides were incubated at 4° C. overnight with 1:100 dilution of primary antibody directed against PCNA and p-EGFR followed by a 30-min incubation in biotinylated goat antimouse secondary antibody. Slides were then incubated with streptavidin peroxidase and visualized using the DAB chromogen (Lab Vision Corp., Freemont, Calif.).

DNA Microarray

DNA microarray analysis of gene expression was done as described by the Brown and Derisi Labs (available at their microarray protocol website, currently at www.microarrays.org/protocols.html). The sequence-verified cDNA clones on the human cDNA microarray are available from Research Genetics (www.resgen.com). Purified PCR products, generated using the clone inserts as template, were spotted onto poly-L-lysine coated microscope slides using an Omnigrid robotic arrayer (GeneMachines, Ca) equipped with quill-type pins (Majer Scientific, Az).

Cells exposed to radiation ±24 hour pretreatment to erlotinib were solubilized and homogenized in Trizol (Invitrogen) and total RNA was isolated according to the manufacturers instruction and integrity was tested. Once isolated, mRNA was used as a template for cDNA generation using reverse transcriptase (RT). Inclusion of amino allyl-dUTP in the RT reaction allowed for subsequent fluorescent labeling of cDNA using mono-functional NHS ester dyes (as described at www.microarrays.org/protocols.html). In each experiment, fluorescent cDNA probes were prepared from an experimental mRNA sample (Cy5-labeled) and a control mRNA sample (Cy3-labeled) isolated from untreated cells. The experimental cDNA sample was coupled to a monofunctional Cy5 NHS-ester and the reference cDNA sample to a Cy3 NHS-ester (Amersham). The labeled probes were then hybridized to 20K human cDNA microarrays. Fluorescent images of hybridized microarrays were obtained using a GenePix 4000A microarray scanner (www.axon.com, Axon Instruments, Ca). The Cy5/Cy3 ratio was collected and the data sets for each experiment were queried for genes that were differentially expressed in the drug treated versus control cell lines (ratios greater than 1.5 or less than 0.75. The data sets from individual analyses were then visualized using the TreeView Program. Identified genes were subsequently categorized using the web based gene ontology program FatiGO (Al-Shahrour, F., et al. (2004) Bioinformatics 20:578-580: details also at website at http://fatigo.bioinfo.cnio.es/).

Quantitative Real Time PCR

To further validate the microarray findings, we performed quantitative real-time PCR (QPCR) using the SYBR green dye as previously described (Kleer, C. G., et al. (2003) Proc. Natl. Acad. Sci. USA 100(20):11606-11611). Briefly, 1 μg of total RNA isolated from each sample was reverse transcribed into first strand cDNA. Threshold levels were set for each experiment during the exponential phase of the PCR reaction using the SDS v 1.7 software (Applied Biosystems), and the quantity of DNA in each sample was calculated by interpolating its Ct value versus a standard curve of Ct values obtained from serially diluted cDNA from a mixture of all of the samples using Microsoft Excel. All standard curves had R2 values ≧0.99 over three orders of magnitude. The calculated quantity of the target gene for each sample was then divided by the average calculated quantity of the housekeeping genes glyceraldehyde-3 phosphate dehydrogenase (GAPD) and hydroxymethylbilane synthase (HMBS) corresponding to each sample to give a relative expression of the target gene for each sample. All oligonucleotide primers were synthesized by Integrated DNA Technologies. Primers for HMBS and GAPD were as described (Vandesompele, J., et al. (2002) Genome Biol.;3(7):RESEARCH0034). Oligonucleotide primers for CXCL1, Il-1β, and Egr-1 are available upon request. All experiments were performed in duplicate.

Statistics

The effects of erlotinib and/or radiation on growth inhibition in xenograft studies were assessed by multiple regression analysis using the PROC GLM procedure in SAS (Version 8, SAS Institute, Inc., Cary N.C., 1999). Combination studies determining apoptosis induction were evaluated using Student's t test with the resultant P value representing a two-sided test of statistical significance.

Results

Cell cycle kinetics.

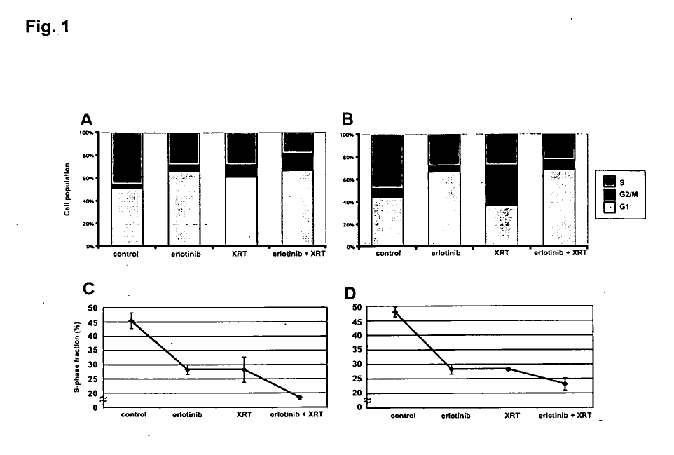

The capacity of erlotinib to inhibit cell cycle progression and to modulate interaction with radiation was evaluated via flow cytometry. The effect of erlotinib on cell cycle phase distribution for the UM-SCC6 and H226 cell lines is summarized in FIG. 1. Erlotinib and ionizing radiation induced accumulation of cells in G1 and G2, respectively, and reduced the number of cells in S phase. When combined with radiation, erlotinib promoted a further reduction in the S phase fraction. This impact of erlotinib on cell cycle phase distribution may contribute to enhanced radiosensitivity as described below.

Erlotinib Enhances Radiation-Induced Apoptosis.

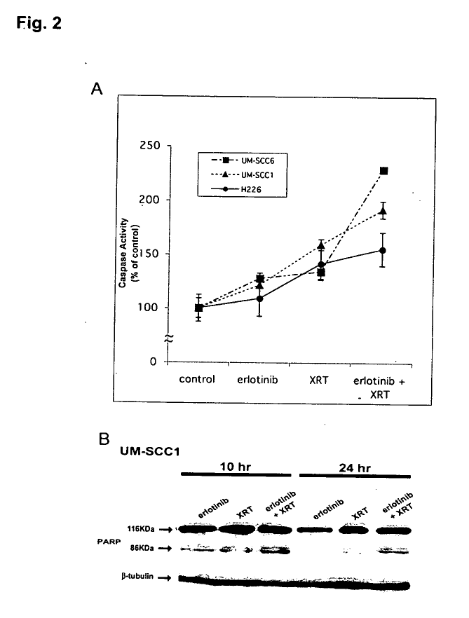

We further evaluated whether mechanisms of interaction between erlotinib and radiation involve cell killing mediated by apoptosis in HNSCC and NSCLC cell lines. As shown in FIG. 2a, erlotinib (1 μM) and radiation alone (6 Gy) induced a 10-25% and 25-50% increase in apoptosis, respectively, as determined by caspase activity. However, combined treatment with radiation and erlotinib resulted in an additive increase in apoptosis in H226 and UM-SCC1 cells, and a supra-additive increase in apoptosis induction (p<0.05) in UM-SCC6 cells. The enhancement of radiation-induced apoptosis by erlotinib was further confirmed using western blot analysis to determine cleavage of the death substrate, poly(ADP-ribose) polymerase (PARP). As shown in FIG. 2b, 10 and 24 hours following treatment, erlotinib and radiation alone induced modest PARP cleavage. Further increase in PARP cleavage is demonstrated when erlotinib is combined with radiation in UM-SCC1 cells. This enhancement in PARP cleavage with the erlotinib/radiation combination is even more pronounced in the UM-SCC6 cells (data not shown).

Erlotinib Inhibits Radiation-Induced Activation of EGFR.

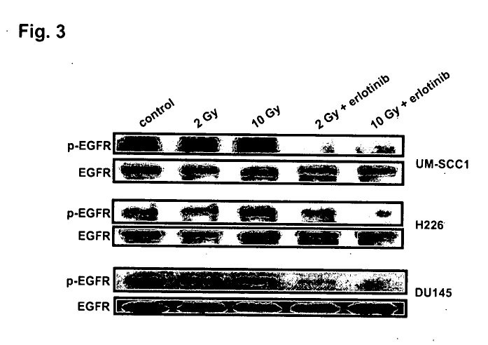

Treatment with ionizing radiation can induce the EGFR proliferative pathway by the release of TGF-α and activation of the EGFR tyrosine kinase. This effect has been proposed to represent a central mechanism for accelerated cellular repopulation during radiation treatment (Schmidt-Ullrich, R. K., et al. (1997) Oncogene 15(10):1191-1197). As depicted in FIG. 3, increased EGFR-autophosphorylation was confirmed following radiation exposure (2 and 10 Gy) in three distinct cell lines (H&N, lung, prostate). This radiation-induced activation of EGFR phosphorylation was profoundly inhibited by pretreatment exposure of tumor cells to 1 μM erlotinib for 24 hrs in culture.

Erlotinib Attenuates Radiation Induced Expression of RAD51.

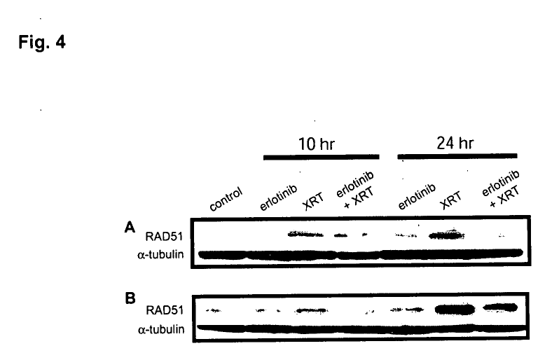

The repair protein Rad51 represents a central component of homologous recombination during DNA repair (Chen, G., et al. (1999) J. Biol. Chem. 274(18):12748-12752; Yuan, Z. M., et al. (1998) J. Biol. Chem. 273(7):3799-3802). Inhibition of Rad51 expression has been shown to correlate with increased radiation sensitivity (Ohnishi, T., et al. (1998) Biochem. Biophys. Res. Commun. 245(2):319-324). To examine the effect of erlotinib on Rad51 expression following radiation exposure, H226 and UM-SCC6 cells were exposed to radiation±pre-treatment with erlotinib. As depicted in FIG. 4, both cell lines demonstrate little to no detectable baseline Rad51 expression. An increase in Rad51 expression was demonstrated following radiation exposure in a time dependent manner (10 and 24 hrs). This increase in Rad51 expression was attenuated significantly when cells were pretreated with 1 μM erlotinib for 24 hrs.

Erlotinib Enhances Radiosensitivity.

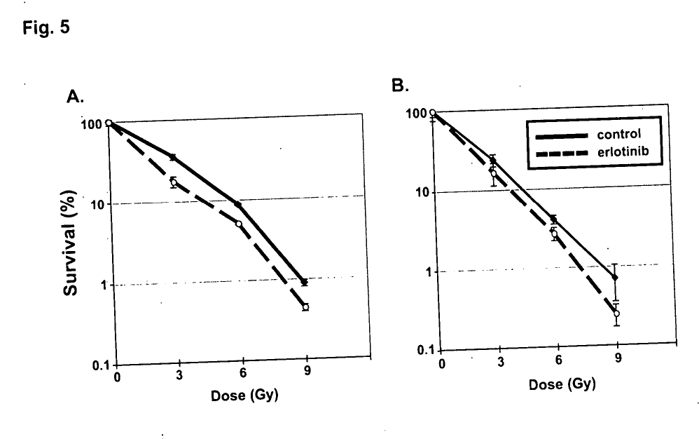

To examine the potential usefulness of combining erlotinib with radiation therapy in human carcinoma cell lines, experiments were conducted to evaluate the influence of erlotinib on clonogenic survival. FIG. 5 depicts clonogenic survival curves for UM-SCC1 and H226 cell lines exposed to erlotinib prior to radiation exposure. These results demonstrate that treatment with erlotinib before radiation induced modest but consistent radiosensitization as manifested by a reduction in clonogenic survival compared with controls in UM-SCC1 at 3, 6, and 9 Gy (p<0.01) and in H226 at 6 and 9 Gy (p<0.05).

Erlotinib Augments In Vivo Tumor Response of NSCLC and SCC Xenografts to Radiation.

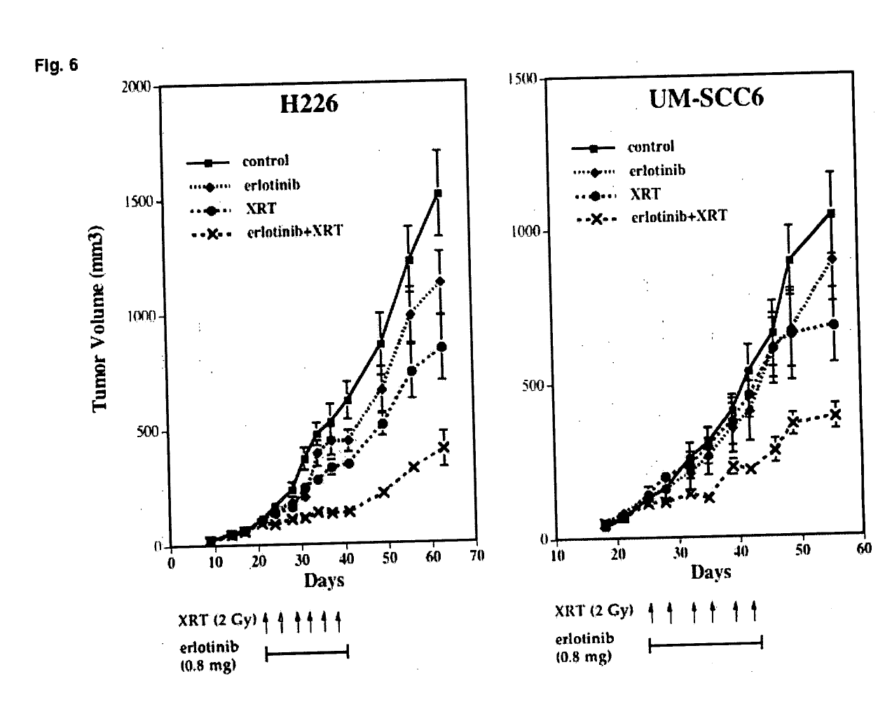

Human NSCLC (H226) and SCC (UM-SCC6) cell lines were inoculated s.c. into female athymic mice and allowed to grow for 10 days before randomization into four groups. Ten days was the time interval required for xenografts to reach ˜20 mm³ in volume. As shown in FIG. 6, treatment with radiation alone or erloltinib alone produced modest inhibition of tumor growth in both H226 and UM-SCC6 xenografts. When combined with radiation, erlotinib enhanced the tumor growth inhibition profile over the 55-day observation period. Statistical analysis confirmed this tumor growth inhibition to be synergistic in the H226 and additive in the UM-SCC6 xenografts (p<0.05).

In Vivo Expression of PCNA and p-EGFR.

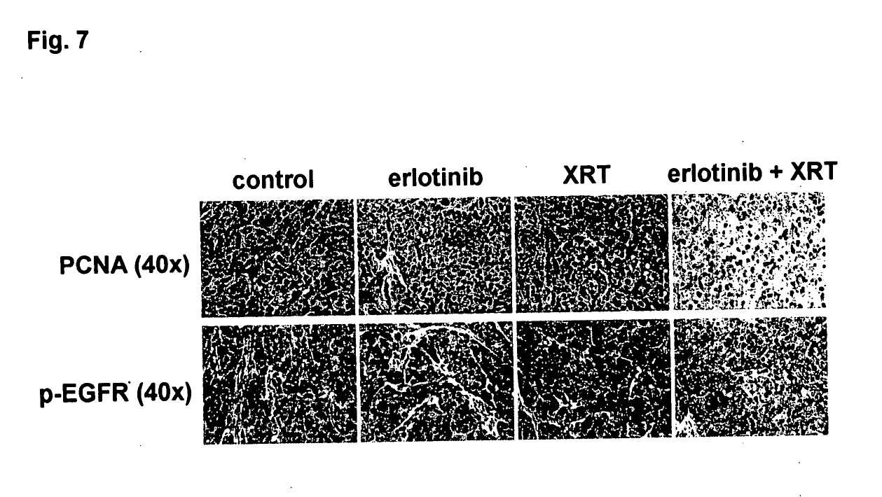

The expression of markers of tumor proliferation (PCNA) and activated EGFR (p-EGFR) were examined in H226 tumor xenografts. Immunohistochemical staining with PCNA demonstrated the number of proliferating cells to be largest in the control group, intermediate in the groups receiving single modality treatment with either radiation or erlotinib, and smallest in the combined treatment group. Immunostaining for p-EGFR demonstrated similar activity in the control and radiation treated groups, with marked inhibition in the combined erlotinib/radiation group. (FIG. 7) Taken together, these results complement in vitro data which demonstrates the capacity of erlotinib to modulate cellular proliferation, apoptosis, and EGFR signaling activation as shown in FIGS. 1-3.

Microarray Analysis.

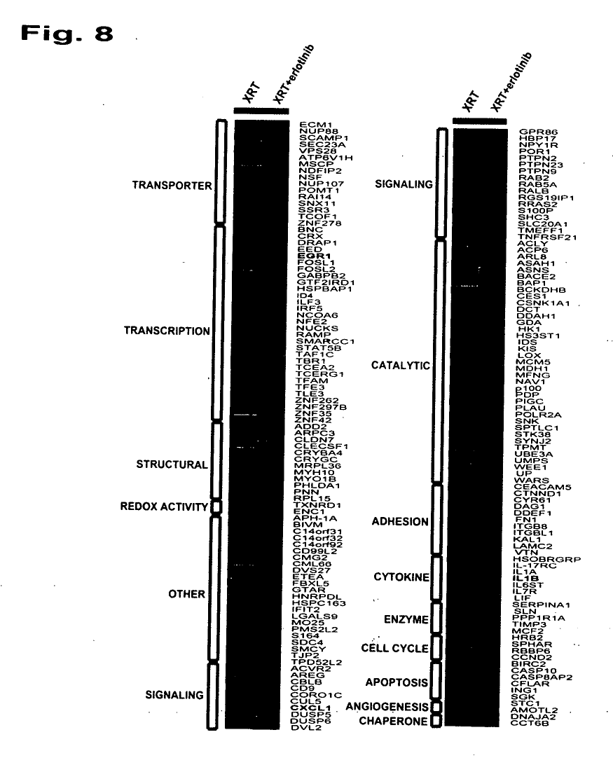

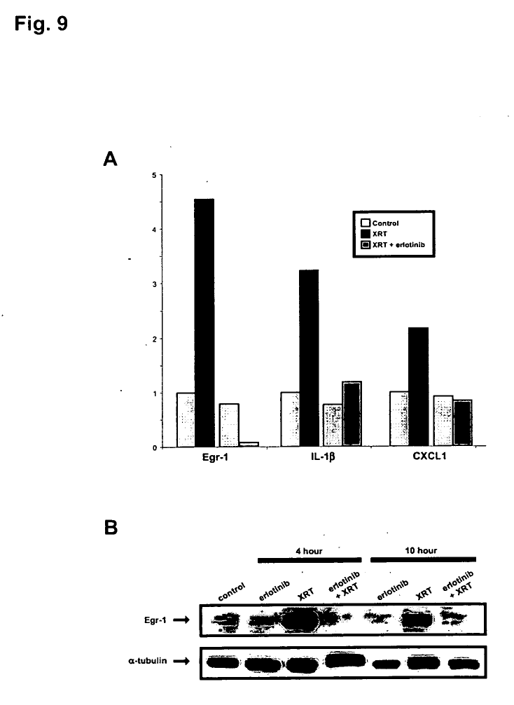

To identify a cohort of genes linking EGFR signaling with radiation response, we used a 20,000 element (20K) cDNA microarray consisting of known, named genes as well as numerous expressed sequence tags (ESTS) (Dhanasekaran, S. M., et al. (2001) Nature 412(6849):822-826). Initial experimentation was performed on UM-SCC6 cells to determine the temporal relationship of gene expression following exposure to radiation. These preliminary studies (data not shown) identified the largest cohort of differentially regulated genes to emerge approximately 24 hours after exposure to radiation. Subsequent array studies were therefore performed on UM-SCC6 cells 24 hours after exposure to radiation±pretreatment with erlotinib. We identified a diverse set of differentially regulated genes (ratios greater than 2 or less than 0.5) involving 14 functional classes (FIG. 8). We validated these DNA microarray findings for a select cohort of genes which may influence the radiosensitization capacity of erlotinib, including Egr-1, CXCL1, and IL-10 (FIG. 9). These validation studies confirmed a potent radiation-induced enhancement of Egr-1, CXCL1, and IL-1β expression which was inhibited by pretreatment exposure to erlotinib.

Discussion

This study characterizes the capacity of the EGFR TKI erlotinib to modulate radiation response in human carcinoma cell lines and xenografts. These results augment emerging preclinical data demonstrating a favorable anti-tumor interaction between EGFR inhibitory agents and radiation. The potential significance of this favorable interaction recently realized a major clinical milestone with results from a Phase III trial in advanced H&N cancer patients. This international study demonstrated a near doubling of median survival for patients treated with the EGFR inhibitor cetuximab over that achieved with radiation alone (Bonner, J. A., et al. (2004) J. Clin. Oncol., 22:(14S) (July 15 Supplement)). There was a statistically significant improvement (log rank p=0.02) in locoregional disease control (8% at 2 yrs) and overall survival (13% at 3 yrs) favoring the cetuximab arm. These pivotal results will stimulate new clinical trials that incorporate EGFR inhibitors in combination with radiation for a variety of cancer types in which radiation plays a central treatment role. Maximizing potential clinical gains in future trials may benefit from an improved understanding of specific mechanisms underlying these favorable EGFR/radiation interactions.

In parallel with previous reports regarding the EGFR monoclonal antibody cetuximab (Erbitux™, C225) and the TKI gefitinib (Iressa™, ZD1839), the magnitude of radiosensitization with erlotinib appears magnified in the in vivo setting (Huang, S. M., et al. (1999) Cancer Res. 59(8):193540; Huang, S. M., and Harari P M. (2000) Clin. Cancer Res. (6):2166-74). The current data suggests potential explanations for this enhanced effect at the level of cell cycle kinetics, accelerated cellular repopulation, and DNA damage repair. In addition, preliminary cDNA microarray studies suggest several angiogenic factors, cytokines, structural and adhesion proteins that are modulated by erlotinib, and may play a role in the enhanced in vivo radiation response.

Previous reports have suggested the capacity of EGFR inhibition to interfere with the activity or localization of DNA-PK, which plays a central role in DNA double strand breaks (DSB) repair (Bandyopadhyay, D., et al. (1998) J. Biol. Chem. 273(3):1568-1573). The current study further supports a relationship between EGFR signaling and DNA damage/repair by linking erlotinib with the DNA damage repair protein, Rad51. Rad51 represents a key protein in homologous recombination during DNA DSB repair (Chen, G., et al. (1999) J. Biol. Chem. 274(18):12748-12752; Yuan, Z. M., et al. (1998) J. Biol. Chem. 273(7):3799-3802). Attenuation of Rad51 expression has been shown to enhance radiation sensitivity (Ohnishi, T., et al. (1998) Biochem. Biophys. Res. Commun. 245(2):319-324) and Rad51 over-expression by tumor cells suggests that this represents a worthy molecular target for tumor radiosensitivity modulation (Raderschall, E., et al. (2002) Cancer Res. 62(1):219-225). The protein expression data in FIG. 4 demonstrates the capacity of erlotinib to attenuate radiation induced expression of Rad51. Although a novel finding, similar interactions have been identified with other tyrosine kinase signaling pathways, including bcr-abl (Yuan, Z. M., et al. (1998) J. Biol. Chem. 273(7):3799-3802; Russell J. S., et al. (2003) Cancer Res. 63(21):7377-7383) and insulin-like growth factor (Trojanek J., et al. (2003) Mol. Cell. Biol. (21):7510-7524). Differential cell cycle phase sensitivity to the cytotoxic effects of radiation is well established, with S phase more resistant and G2/M more sensitive to radiation (Hall E J. (2000) Radiobiology For the Radiologist. 5 ed. Philadelphia: Lippincott Williams & Wilkins). In the current study, independent exposure of cells to erlotinib or radiation elicits a characteristic G1 and G2/M phase arrest, respectively. Erlotinib exposure alone reduces the percentage of cells in the radiation resistant S phase fraction. When combined with single fraction radiation, erlotinib precipitates a further decrease in the S phase fraction. Subsequent radiation treatments might therefore be expected to encounter a higher percentage of cells in more radiation sensitive phases of the cell cycle. This cell cycle kinetics interaction might explain the enhanced radiation response demonstrated using in vivo models, which used fractionated radiation regimens.

Cytotoxic therapies can trigger surviving tumor clonogens to divide more rapidly than before, a phenomenon termed accelerated repopulation. This proliferation of tumor cells during a radiation treatment course has been well defined as a factor that adversely impacts overall tumor response and ultimate local control (Fowler J. F., et al. (1992) Int. J. Radiat. Oncol. Biol. Phys. 23(2):457-467). A proposed mechanism for accelerated cellular repopulation involves the capacity of ionizing radiation to activate EGFR, which is linked to several critical components of mitogenic and proliferative signaling (Schmidt-Ullrich, R. K., et al. (1997) Oncogene 15(10):1191-1197). In the present study, we demonstrate the capacity of erlotinib to inhibit radiation-induced activation of EGFR signaling (FIG. 3), thereby providing a potential method to combat accelerated repopulation during fractionated radiation.

To further examine of potential EGFR/radiation interactions, preliminary microarray analysis of human tumor cells was performed. These studies identified >100 genes that were differentially expressed (i.e. genes that were significantly up or down regulated) following radiation, and subsequently normalized or reversed by erlotinib pretreatment. The identified genes represent several distinct functional classes involved in diverse oncogenic processes including cell structure, inflammation, adhesion, apoptosis, and tumor angiogenesis. Particular genes were selected which may provide further insight regarding mechanistic synergy between EGFR signaling and radiation response including: Egr-1 and the chemokines IL-1β and CXCL1 which have been linked to NF-κB activation.

Egr-1 encodes a zinc finger transcription factor which can be induced by a variety of stimuli, including growth factors, cytokines, and mitogens. A series of DNA damaging agents have been reported to induce significant upregulation of Egr-1 (Quinones A., et al. (2003) Life Sci. 72(26):2975-2992). Inhibition of Egr-1 expression has been demonstrated to inhibit microvascular endothelial cell replication and migration, microtubule formation, VEGF expression, and tumor angiogenesis (Lucerna M., et al. (2003) J. Biol. Chem. 278(13):11433-11440. Epub Nov. 8, 2002 ; Fahmy R. G., et al. (2003) Nat. Med. 9(8): 1026-1032). Additionally, Egr-1 expression has been demonstrated to increase EGFR expression during hypoxia (Nishi H, et al. (2002) Cancer Res. 62(3):827-834). This study demonstrates the capacity of erlotinib to attenute Egr-1 expression following exposure to radiation.

Various interleukins and chemokines are induced and released following exposure to ionizing radiation. These molecules can participate either directly or indirectly in subsequent radiation response (Fornace A. J., et al. (2001) Radiation Therapy. In: Straus M., editor. The Molecular Basis of Cancer. 2nd ed. Philadelphia: W.B. Saunders Company; p. 423-465). We identified two cytokines, IL-1β and CXCL1, which may contribute to tumor growth and survival during radiation and may strengthen a link between EGFR signaling and the pro-survival signaling system, nuclear factor-kappa B (NF-κB) (Kapoor G. S., et al. (2004) Mol. Cell. Biol. 24(2):823-36; Biswas, D. K., et al. (2000) Proc. Natl. Acad. Sci. U.S.A. 97(15):8542-7; Habib, A. A., et al. (2001) J Biol. Chem. 276(12):8865-8874. Epub Dec. 14, 2000).

Several reports indicate that IL-1β induced by radiation can afford radioprotection (Fomace A. J., et al. (2001) Radiation Therapy. In: Straus M., editor. The Molecular Basis of Cancer. 2nd ed. Philadelphia: W.B. Saunders Company; p. 423-465) as well as stimulate tumor cell proliferation, angiogenesis, and invasion (Giavazzi, R., et al. (1990) Cancer Res. 50(15):4771-4775; Song, X, et al. (2003) J. Immunol. 171(12):6448-6456; Dinarello, C. A. (1996) Blood 87(6):2095-2147). IL-1β exerts many biological effects by activating the transcription factor NF-κB, which in turn regulates the expression of a variety of inflammatory and oncogenic processes (Jung, Y. J., et al. (2003) FASEB J. (14):2115-2117. Epub Sep. 4, 2003 ). The capacity of erlotinib to attenuate radiationinduced transcription of IL-1β may therefore decrease NF-κB DNA binding activity. This link between ErbB signaling and NF-κB activity has recently been reported using the ErbB inhibitors trastuzumab and cetuximab (Guo, G., et al. (2004) Oncogene 23(2):535-545; Sclabas, G. M., et al. (2003) J. Gastrointest. Surg. (1):37-43).

The CXC chemokine CXCL1, previously designated as melanoma growth stimulatory activity/growth related protein (MGSA/GRO), has recently been characterized as one of many chemokines involved in radiation response (Van der Meeren, A., et al. (2003) Radiat. Res. 160(6):637-646). CXCL1 has been shown to play an important role in tumorgenesis and angiogenesis and its overexpression has been associated with tumor progression (Dhawan, P., and Richmond A. (2002) J. Biol. Chem 277(10):7920-2928. Epub Dec. 28, 2001). Increased expression of CXCL1 has been attributed to constitutive activation of NF-κB through MAP kinase signaling (Dhawan, P., and Richmond A. (2002) J. Biol. Chem 277(10):7920-2928. Epub Dec. 28, 2001). Therefore, the capacity of erlotinib to inhibit MAP kinase signaling and to influence NF-κB activation may reflect a mechanistic interaction linking EGFR signaling with CXCL1 expression.

### CONCLUSIONS

These preclinical results identify potential cellular mechanisms whereby EGFR signaling inhibition can enhance tumor radiation response. The first Phase III clinical trial to examine the combination of EGFR inhibitor plus radiation (H&N cancer) indicates favorable outcome with increased patient survival over that achieved with radiation alone (Bonner, J. A., et al. (2004) J. Clin. Oncol., 22:(14S) (July 15 Supplement)). The newly reported Phase III clinical trial confirming survival extension in refractory NSCLC patients receiving erlotinib (Shepherd, F. A., et al. (2004) J. Clin. Oncol., 22:(14S) (July 15 Supplement)) also suggests opportunities to explore combination approaches in lung cancer, where radiation plays a central treatment role. Systematic efforts to define specific cellular and molecular mechanisms for this favorable interaction of EGFR signaling inhibition with radiation should assist in the design of future clinical trials.

Incorporation by Reference

All patents, published patent applications and other references disclosed herein are hereby expressly incorporated herein by reference.

Equivalents

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method for treating tumors or tumor metastases in a patient, comprising administering to the patient a therapeutically effective amount of the EGFR kinase inhibitor erlotinib, combined with treating the patient simultaneously or sequentially with radiation therapy.

2. The method of claim 1, wherein the patient is a human that is being treated for cancer.

3. The method of claim 1, wherein the treatment of the patient with radiation therapy is sequential.

4. The method of claim 1, wherein erlotinib is administered to the patient by parenteral or oral administration.

5. The method of claim 1, additionally comprising administering one or more other anti-cancer agents.

6. The method of claim 5, wherein the other anti-cancer agents are selected from an alkylating agent, cyclophosphamide, chlorambucil, cisplatin, carboplatin, oxaliplatin, busulfan, melphalan, carmustine, streptozotocin, triethylenemelamine, mitomycin C, an anti-metabolite, methotrexate, etoposide, 6-mercaptopurine, 6-thiocguanine, cytarabine, 5-fluorouracil, capecitabine, gemcitabine, dacarbazine, an antibiotic, actinomycin D, doxorubicin, daunorubicin, bleomycin, mithramycin, an alkaloid, vinblastine, paclitaxel, docetaxel, vinorelbine, a glucocorticoid, dexamethasone, a corticosteroid, prednisone, a nucleoside enzyme inhibitors, hydroxyurea, an amino acid depleting enzyme, asparaginase, topotecan, irinotecan, leucovorin, and a folic acid derivative.

7. The method of claim 1, wherein the tumors or tumor metastases to be treated are lung cancer tumors or tumor metastases.

8. The method of claim 1, wherein the tumors or tumor metastases to be treated are head and neck cancer tumors or tumor metastases.

9. A method for the treatment of cancer, comprising administering to a subject in need of such treatment an effective first amount of the EGFR kinase inhibitor erlotinib, or a pharmaceutically acceptable salt thereof, and an effective second amount of radiation therapy.

10. The method of claim 9, wherein the cancer is a head and neck cancer.

11. The method of claim 9, wherein the cancer is a lung cancer.

12. The method of claim 11, wherein the lung cancer is NSCLC.
